(19) United States
(12) Patent Application Publication
(10) Pub. No.: US 2013/0085149 A1
(43) Pub. Date: Apr. 4, 2013

(54) OSMOLYTE-CONTAINING PREPARATION FOR THE TREATMENT OF DRY MUCOUS MEMBRANES

(71) Applicant: Bitop AG, Witten (DE)

(72) Inventor: Hans Bernd Schmittmann, Velbert (DE)

(73) Assignee: Bitop AG, Witten (DE)

(21) Appl. No.: 13/625,793

(22) Filed: Sep. 24, 2012

(30) Foreign Application Priority Data
Oct. 31, 2007 (DE) 1020070523809

(51) Int. Cl. A61K 31/505

(52) U.S. Cl. CPC A61K 31/505
USPC 514/256

(57) ABSTRACT

The present invention relates to osmolyte-containing preparations for the local treatment of dry mucous membranes. It describes the use of osmolytes for the production of a medicament, medical product or cosmetic product for the prevention, therapy and/or care of dry mucous membranes. The present invention relates to topical compositions based on osmolytes to which sodium chloride and/or moisturizers can optionally be added. The group of osmolytes proposed by the invention embraces various low-molecular substances, in particular ectoine, homoectoine, hydroxyectoine, di-myo-inositol phosphate (DIP), cyclic 2,3-diphosphoglycerate (cDPG), 1,1-di-glycerol phosphate (DGP), β-mannosylglycerate (Firoin), β-mannosylglyceramide (Firoin-A), di-mannosyl di-inositol phosphate (DMIP), glucosylglycerol and/or a derivative, e.g. an acid, salt or ester, of these compounds.

### CROSS REFERENCE TO RELATED APPLICATIONS

This application is a continuation of and claims the benefit of prior U.S. application Ser. No. 12/740,021 filed on Jun. 29, 2010, which is incorporated by reference herein in its entirety.

Aside from smell perception the nose also fulfills other important duties: it cleans the breathing air by removing small particles, heats the inhaled air up to body temperature and humidifies it. In this manner pathogenic factors are eliminated and the exchange of gas in the lungs is most favorably prepared. However, this can only work properly if the nasal mucosa is capable of humidifying the breathing air sufficiently. In the event air is particularly dry which is the case during winter time or in air-conditioned rooms where air humidity may be less than 5 g of water per cubic meter of air, the capacity of the nasal mucosa soon proves insufficient. In this case symptoms such as rhinitis sicca (dry nose) may be experienced accompanied by itching, burning sensation, eczema and crust formation. Sometimes nose bleeding may occur and the nasal passage may often be clogged up even without a common cold having been caught. These symptoms alone can be very unpleasant. However, even more adverse consequences of a dry nose arise from its loss of function: The excessively dry, too cold and unfiltered air sooner or later will carry disease-causing organisms into the now unprotected respiratory tract.

The “dry nose syndrome” which may manifest itself in the form of rhinitis sicca or atrophic rhinitis is to be considered a serious medical problem. This may also occur due to a side effect of a certain medicinal treatment of the nose and when people stay in air-conditioned rooms repeatedly or for a prolonged period of time. Additionally, many patients suffering from a dry mucous membrane of the nose are heavy smokers (cigarette abuse).

More often than not, an aqueous, isotonic common salt solution is the agent of choice to be applied when treating a dry nose. However, a treatment applying an agent in spray form may not always produce satisfactory effects and must be repeated quite often. In comparison with other nasalia preparations of higher viscosity offer characteristic benefits: Other than water-containing nose drops or sprays they remain longer on the nasal mucous lining and for that reason have a more caring and beneficial effect. However, administering aqueous viscous preparations also has a drawback in that an unpleasant crust builds up after the water in the viscosity producing agent has evaporated. Moreover, a most serious side effect or problem is linked with the subjective impression of a “dry nose” and hardly any significantly satisfactory treatment is presently available to remedy this situation. Patent application DE 43 04 893 has proposed polyols (e.g. glycerine, glycol 300-1000, polypropylene glycol 300-1000) in an inert polymer, preferably of a non-Newtonian rheological profile.

It has been found in this context that high viscosity is needed to bring about satisfactory clinical effects. On the other hand, the use of viscous preparations usually enables only the nasal vestibule to be reached so that deeper areas of the nasal mucosa can only be treated insufficiently.

Application of mineral oils in the nose is considered a matter of concern nowadays because they may lead to the formation of granulomas (nodules) in the nasal mucosa. Such nodules develop when the nasal mucosa tries without avail to remove the inert paraffin by resorptive processes. Moreover, inhaling paraffin may also give rise to the formation of intrapulmonary granulomas.

On the other hand, a repeated application of vasoconstrictory or nasal mucosa decongestant additives (sympathomimetic substances) often results in the mucous nasal linings to become desiccated which may lead to inflammatory irritations. These side effects may entail major risks of infection since mucous membranes in desiccated and inflamed condition will no longer be capable of performing their protective and filtering functions satisfactorily so that disease-causing organisms may enter the anatomical airway. Therefore, additions of pantothenol or pantothenetic acid or acidic glycosamine glycans are described in publications DE 195 41 919, DE 195 49 421 and DE 103 56 248. Nevertheless, this could do no more than lessen the above mentioned drawbacks associated with prior-art techniques. The disclosed compositions do not contain additives having anti-inflammatory effects. For that reason, the alleviated inflammatory irritations are thought to be due to the improved humidification of the mucous nasal membrane. To counteract the generally known side effects of sympathomimetic substances more efficiently utility patent publication DE 20 2006 005 924 recommends the use of myrrh. According to the composition disclosed in that publication myrrh shall produce anti-inflammatory, antiphlogistic effects. Adding zinc compounds enables the affected cells to better neutralize free radicals and thus assists the effects produced by myrrh. It thus follows that only a common salt solution counteracts the desiccation of the mucous membranes.

It is thus the objective of the present invention to provide a preparation which is suitable for the prevention, therapy and/or care of dry mucous nasal membranes and, in particular, overcomes the disadvantages of the prior-art approaches elucidated hereinbefore.

Another objective of the present invention is to provide a preparation which does not require sympathomimetic substances with vasoconstrictory and/or nasal mucosa decongestant properties. Alternatively, the inventive preparation shall be capable of alleviating the desiccation and inflammatory irritation of the nasal mucosa which are typical side effects of sympathomimetic substances.

A desirable preparation must therefore satisfy the following requirements:

a) Higher salt concentrations no longer moisten the mucous membranes but, on the contrary, they even cause water to be extracted. Compositions which do not extract water, even when they are of higher concentration, and, moreover, exhibit a higher physiological compatibility are thus considered preferable.

b) Moistening brought about by an optional common salt/solar salt solution or seawater-containing solution must be facilitated and the above described side effects reduced.

c) The anti-inflammatory additives must be better adapted to a preparation which may contain salt.

d) The additives should enable further pharmacological active agents to be stabilized if necessary and/or alleviate their side effects.

Unexpectedly, the inventor has now found that these problems can be solved by providing a preparation or formulation on the basis of osmolytes or derivatives of osmolytes to which, optionally, sodium chloride and/or moisturizers may be added. As moisturizers scleroglucanes (for example Tinocare) are of special advantage here. Derivatives in this context are, in particular, the relevant acids, salts or esters.

The preparation may also serve as vehicle for dispensing a medicament.

The term “preparation” or “formulation” or a similar term as it is used in the framework of the present invention has a very broad meaning and shall not only embrace pharmaceutical preparations or pharmaceutical products as such but also so-called medicinal products or the like as well as cosmetics.

Osmolytes and compatible solutes are natural active agents that enable human skin to be protected against harmful environmental influences without producing side effects (e.g. M. F. Roberts, “Organic compatible solutes of halotolerant and halophilic microorganisms”, Saline Systems 2005, 1: 5, http://www.saline systems.org/content/1/1/5). For example, the osmolytes ectoine and hydroxyectoine protect cell structures of human skin and their genetic material against the detrimental effects of UV radiation exposure and other forms of environmental stress. As a result of cell protection through ectoines the immune response of the skin cells and thus the skin's self protection mechanism is maintained for a longer period of time and thus prevents permanent skin damage. Due to their protective function ectoines delay inflammatory reactions of the skin. That ectoines possess these properties has been proven through many application studies, and ectoines in various conventional cosmetic products have already been put on the market.

Ectoines for the production of medicinal products are mentioned in EP 0 887 418, but without specifying the relevant medicinal products. Ectoine-containing pharmaceutical preparations containing at least one protein-containing substance (WO 00/76528) or one pharmaceutically permissible carrier (EP 0 553 884) are known as well.

Ectoines as natural cell protective agent are won from extremophilic microorganisms. Extremophilic microorganisms count among the oldest life forms on earth and are optimally adapted to most adverse environmental conditions such as extreme temperatures (even above 100° C.) or high salt content (200-300 g/l). Their natural habitats are, for example, salines, hot springs or undersea volcanoes. Extremolytes are indispensable for the protection of various extremophilic microorganisms against stress factors such as cold, heat, salt, UV radiation or radicals. The group of osmolytes includes in particular 1,4,5,6-tetrahydro-2-methyl-pyrimidine-4-carboxylic acid (ectoine), 4,5,6,7-tetrahydro-2-methyl-1H-[1,3]-diazepine-4-S-carboxylic acid (homoectoine), S,S-β-hydroxy-1,4,5,6-tetrahydro-2-methyl-pyrimidine-4-carboxylic acid (hydroxyectoine), di-myo-inositol phosphate (DIP), cyclic 2,3-diphosphoglycerate (cDPG), 1,1-di-glycerol phosphate (DGP), β-mannosylglycerate (firoin), β-mannosylglyceramide (firoin-A), di-mannosyl-di-inositol phosphate (DMIP) and glucosylglycerol. As compared to the polyols mentioned in patent application DE 43 04 893 glycerine derivates offer the advantage of having a by far better physiological compatibility.

Typically, the osmolytes have a concentration ranging between 0.001 and 50% w/w, preferably 0.05 to 20% w/w, in particular 0.1 to 10% w/w based on the total weight of the composition.

As mentioned earlier, the composition may also include sodium chloride in the form of common salt, solar salt or seawater. Based on one liter of the composition the content, for example, amounts to 0.5 to 20 g, in particular 1 to 10 g, preferably 2 to 8 g, especially preferred 5 to 7 g. When using seawater the salt content can optionally be re-adjusted by means of common salt/solar salt.

The consistency of the preparation according to the invention may be liquid or viscous to semisolid. For example, the inventive formula may be provided in the form of an ointment, cream or gel for application into the nose or, preferably, as solution or dispersion to be dripped or sprayed into the nose or as irrigation solution.

As carrier for liquid pharmaceutical forms especially aqueous systems with or without buffer have proved expedient. As carrier substances for viscous or semisolid preparations, which may be ointments, creams or gels for example, paraffin hydrocarbons, Vaseline, wool wax products and other pharmaceutically usable, viscosity-increasing base materials are suited for example; for hydrophilic gels, for example, water, glycerine or sorbite, gelatinized by means of, for example, polyacrylic acid, cellulose derivatives, starch or traganth. Especially with salt-containing compositions the thickening method is to be selected such that to the extent possible the preparation is prevented from entering the pharynx.

Aside from active and carrier agents/substances and, as the case may be, existing emulgators, the inventive preparation may yet contain other unobjectionable and, in relation to the active agents compatible pharmaceutical auxiliary substances and/or additives, such as for example filler, diluting, binding, wetting, stabilization, coloring, buffering, odorous and/or preservation substances.

Of special significance in this context are additions of tea or tea extracts as well as aloe vera. As natural wetting agents saponines offer a variety of application possibilities. Due to their surface-active properties they are frequently employed in cosmetics and foodstuff. From a physiological viewpoint, their permeability-increasing and thus resorption-increasing effects on membranes are known. Moreover, the composition according to the invention may contain in customary concentration microbiologically active chemical compounds, such as for example preservation substances, antiseptics or manuka oil to improve the microbial stability. Furthermore, the inventive composition or formulation may also contain one or several pharmacologically effective substances. For example, sorbates, benzoates or manuka oil may be employed as preservation agents. Typically, the concentration in this case is in a range of between 0.02 and 5% w/w in relation to the total weight of the composition.

Additionally, the preparations may be provided with an pH buffering system to enable a certain pH value to be adjusted. This may in particular be a buffering system on the basis of citrate/citric acid or on phosphate-/hydrogen phosphate basis.

The composition may serve also as vehicle for dispensing a medicament. Active agents additionally contained in the composition may thus be stabilized and/or their side effects lessened. Moreover, by administering the osmolytes as proposed by the invention together with other active agents synergistic effects can be produced with positive results. For example, the decongestant effects of oxymetazoline, xylometazoline or tramazoline can be combined with the effects of the osmolytes. In particular, the effects of the osmolytes can be combined with the anti-inflammatory effects of other substances, such as for example dexpanthenol or panthenol. Another conceivable combination is with antihistamine drugs such as azelastine or cromoglicic acid. Still another combination can be brought about with viscosity-increasing substances such as hydroxypropyl methylcellulose, hyetellose, hypromellose or hyaluronic acid or with moistening substances such as sesame oil.

In addition to the preparation/composition itself the invention also relates to the use of osmolytes for the production of an agent to be employed for the prophylactic and/or curative topical treatment of dry mucous membranes, in particular of nasal mucous membranes. In this manner a secretion build-up as well as the occurrence of desiccation and inflammatory irritations of the mucous membranes can be avoided. The treatment of dry mucous membranes also serves to reduce the formation of edemas and improve the nasal ventilation, especially ventilation of the paranasal sinuses and tubes.

According to the invention the provision of an inhalation device in the form of a filled inhalator for liquid compositions as proposed by the invention is also possible.

The composition can be manufactured in a manner known per se. For example, this may be achieved by mixing or dissolving the active agents of pharmacologically effective concentrations, the auxiliary substances and/or additives as well as any further pharmacologically effective substances in the envisaged carrier medium.

The following exemplary embodiments shall only serve to provide elucidation of the present invention but are not be intended to be exhaustive or comprehensive.

### EXAMPLE 1 [ECTOINE, ISOTONIC IN WATER]

Purified water is filled into a suitable agitator vessel to approx. 45% of the envisaged final volume. Following this, 3.87% (w/w) of ectoine are added and dissolved by stirring. The solution thus obtained is topped up with purified water to approx. 98% of the final volume and the pH value is adjusted to a pH of 5.5-6.0 by adding 1 N caustic solution/lactic acid (Pural 80). The solution is topped up to the envisaged final volume by adding purified water, then passed through a suitable strainer and filled into bottles which are subsequently provided with a suitable nasal spray pump.

### EXAMPLE 2 [ECTOINE WITH SALT, ISOTONIC IN WATER]

Purified water is filled into a suitable agitator vessel to approx. 45% of the envisaged final volume. Following this, 0.5% (w/w) of ectoine as well as 0.78% (w/w) of common salt or solar salt are added and dissolved by stirring. The solution thus obtained is topped up with purified water to approx. 98% of the final volume and the pH value is adjusted to a pH of 5.5-6.0 by adding 1 N caustic solution/lactic acid (Pural 80). The solution is topped up to the envisaged final volume by adding purified water, then passed through a suitable strainer and filled into bottles which are subsequently provided with a suitable nasal spray pump.

### EXAMPLE 3

Purified water is filled into a suitable agitator vessel to approx. 45% of the envisaged final volume. Following this, 0.5% of ectoine, 0.78% of common salt or solar salt as well as 4.9% of Tinocare SG-L (generic name sclerotium gum) are added and dissolved by stirring. The solution thus obtained is topped up with purified water to approx. 98% of the final volume and the pH value is adjusted to a pH of 5.5-6.0 by adding 1 N caustic solution/lactic acid (Pural 80). The solution is topped up to the envisaged final volume by adding purified water, then passed through a suitable strainer and filled into bottles which are subsequently provided with a suitable nasal spray pump.

### EXAMPLE 4

Purified water is filled into a suitable agitator vessel to approx. 45% of the envisaged final volume. Following this, 0.5% of ectoine, 0.78% common salt, 0.1% saponine Q (DAB 9) as well as 4.8% Tinocare SG-L are added and dissolved by stirring. The solution thus obtained is topped up with purified water to approx. 98% of the final volume and the pH value is adjusted to a pH of 5.5-6.0 by adding 1 N caustic solution/lactic acid (Pural 80). The solution is topped up to the envisaged final volume by adding purified water, then passed through a suitable strainer and filled into bottles which are subsequently provided with a suitable nasal spray pump.

### EXAMPLE 5 [ECTOINE WITH SALT, IN TEA]

Tea (of which 1.50% is chamomile tea or green tea) is filled into a heatable agitator vessel to approx. 45% of the envisaged final volume. The pH value is adjusted to a pH of 5.5-6.0 by adding 1 N caustic solution/lactic acid (Pural 80. Subsequently, 0.5% of ectoine, 2.00% common salt/solar salt, 4.18% Tinocare SG-L, 4.00% active aloe, 1.00% sodium asorbyl phosphate, 0.20% potassium sorbate, 0.10% saponine Q (DAB 9), 0.02% Na-hyaluronate as well as 0.50% glucosaminoglycan are added and dissolved by stirring at a temperature of 45-50° C. The solution thus obtained is blended with 0.50% of guar gum and briefly mixed in a dispersing device to eliminate lumps. The above described tea is used to top up the solution to approx. 98% of the final volume and the pH value is adjusted to a pH of 5.5-6.0 by adding 1 N caustic solution/lactic acid (Pural 80). After a holding time of approx. 24 hours the initial turbidity has vanished to a great extent. The solution is topped up to the envisaged final volume by adding the above-described tea, then passed through a suitable strainer and filled into suitable pipette bottles.

### EXAMPLE 6 [ECTOINE WITHOUT SALT, IN TEA]

Tea (of which 1.50% is chamomile tea or green tea) is filled into a heatable agitator vessel to approx. 45% of the envisaged final volume. The pH value is to be adjusted to a pH of 5.5-6.0 by adding 1 N caustic solution/lactic acid (Pural 80. Subsequently, 0.5% of ectoine, 5.00% Tinocare SG-L, 5.00% active aloe, 0.50% sodium asorbyl phosphate, 0.20% potassium sorbate, 0.20% saponine Q (DAB 9), 0.02% Na-hyaluronate as well as 0.50% glucosaminoglycan are added and dissolved by stirring at a temperature of 45-50° C. The solution thus obtained is blended with 0.48% of guar gum and briefly mixed in a dispersing device to eliminate lumps. The above described tea is used to top up the solution to approx. 98% of the final volume and the pH value is adjusted to a pH of 5.5-6.0 by adding 1 N caustic solution/lactic acid (Pural 80). After a holding time of approx. 24 hours the initial turbidity has vanished to a great extent. The solution is topped up to the envisaged final volume by adding the above-described tea, then passed through a suitable strainer and filled into suitable pipette bottles.

### Application Studies

50 outpatients diagnosed to suffer from rhinitis sicca anterior were examined to ascertain the effectiveness of an inventive ectoine solution. The treatment period was two weeks. The patients were advised to apply the ectoine nasal spray at least 5 times a day.

As main target parameters the subjective affectivity scale of nasal breathing impediment was documented using scores 0 to 12 (0—no, 3—minor, 6—medium, 9—severe and 12—very severe) as per information given by the patients, as well as the extent of crust formation established according to scores 0 to 12 (0—no, 3—minor, 6—medium, 9—severe and 12—very severe). Moreover, as auxiliary target parameters the endonasal deposition of blood, signs of an accompanying pharyngitis, smell nuisance, rhinorrhea, viscosity of secretion and nasal concha hyperplasia were assessed. In this case too a score scale ranging between 0 and 12 was used to quantify the auxiliary target parameters.

After a therapy period of one or two weeks the effectiveness, compatibility and patient compliance was recorded by the examiner using scoring scales 0 to 12 (0—very good, 3—good, 6—sufficient, 9—minor and 12—none/poor).

### Nasal Breathing Impediment as Main Target Parameter:

The following scores were determined:

| | |
|---|---|
| Prior to treatment | 4.6 |
| after one week | 2.76 |
| after two weeks | 1.54 |

The change in nasal breathing impediment was found to be highly significant (p<0.001).

### Crust Formation/Dryness Feeling Inside the Nose as Main Target Parameter:

The following scores were determined:

| | |
|---|---|
| Prior to treatment | 6.2 |
| after one week | 2.16 |
| after two weeks | 1.52 |

The regression was found to be highly significant (p<0.001).

### Blood Deposition as Auxiliary Target Parameter:

The following scores were determined:

| | |
|---|---|
| Prior to treatment | 2.14 |
| after one week | 0.36 |
| after two weeks | 0.36 |

Here again, the change is to be viewed highly significant statistically (p<0.001).

### Pharyngitis as Auxiliary Target Parameter:

The following scores were determined:

| | |
|---|---|
| Prior to treatment | 1.08 |
| after one week | 0.34 |
| after two weeks | 0.16 |

This regression was also found to be highly significant statistically (p<0.001).

### Cacosmia as Auxiliary Target Parameter:

A cacosmia (n=2) has been described in medical diagnostic documentations to have only occurred prior to the treatment. This symptom was described by merely a few patients so that no statistically significant differences could be determined here.

### Rhinorrhea as Auxiliary Target Parameter:

The following scores were determined:

| | |
|---|---|
| Prior to treatment | 1.64 |
| after one week | 1.6 |
| after two weeks | 0.94 |

However, this result was not found to be statistically significant (p=0.248).

### Viscosity of Secretion as Auxiliary Parameter:

The following scores were determined:

| | |
|---|---|
| Prior to treatment | 5.26 |
| after one week | 2.44 |
| after two weeks | 2.00 |

The regression of symptoms was found to be highly significant statistically (p<0.001).

### Assessment of Effectiveness, Compatibility and Patient Compliance:

Score assessment from a medical viewpoint:

| | Effectiveness | Compatibility | Patient compliance |
|---|---|---|---|
| After one week | 3.86 | 2.16 | 2.30 |
| After two weeks | 3.50 | 2.08 | 2.12 |

Patients' assessment of effectiveness and compatibility:

| | Effectiveness | Compatibility |
|---|---|---|
| After 3 days | 4.58 | 2.10 |
| After 6 days | 4.14 | 1.92 |
| After 9 days | 3.46 | 1.60 |
| After 12 days | 3.12 | 1.40 |

The results were found to be highly significant statistically.

When reading the description, further configurations, modifications and variations as well as advantages of the present invention are without difficulty perceptible to and feasible for persons skilled in the art, without leaving the framework or scope of the proposed invention.

1. A method for prophylactic and/or curative topical treatment of dry nasal mucous membranes comprising applying a preparation comprising at least one osmolyte to said membranes,
characterized in that the osmolyte is 1,4,5,6-tetrahydro-2-methyl-pyrimidine-4-carboxylic acid (ectoine), 4,5,6,7-tetrahydro-2-methyl-1H-[1,3]-diazepine-4-S-carboxylic acid (homoectoine), S,S-β-hydroxy-1,4,5,6-tetrahydro-2-methyl-pyrimidine-4-carboxylic acid (hydroxyectoine), di-myo-inositol phosphate (DIP), cyclic 2,3-diphosphoglycerate (cDPG), 1,1-di-glycerol phosphate (DGP), β-mannosylglycerate (firoin), β-mannosylglyceramide (firoin-A), di-mannosyl-di-inositol phosphate (DMIP), glucosylglycerol and/or an acid, salt or ester, of said compounds.

2. (canceled)

3. The method according to claim 1, characterized in that the membranes are dry nasal mucous membranes.

4. The method according to claim 1, characterized in that the preparation contains sodium chloride.

5. The method according to claim 4, characterized in that the preparation contains sodium chloride in an amount of between 0.5 and 20 g based on one liter of the composition.

6. The method according to claim 1, characterized in that the preparation contains a moisturizer, wherein the moisturizer is a scleroglucane.

7. The method according to claim 1, characterized in that the osmolytes have a concentration ranging between 0.001 and 50% w/w based on the total weight of the composition.

8. The method according to claim 1, characterized in that the preparation contains sorbates, benzoates and/or manuka oil of a concentration ranging between 0.02 and 5% w/w as preservation agents.

9. The method according to claim 1, characterized in that the preparation contains aloe vera, tea and/or tea extracts.

10. The method according to claim 1, characterized in that the preparation contains oxymetazoline, xylometazoline, tramazoline, dexpanthenol, panthenol, sesame oil, cromoglicic acid, azelastine, hydroxypropyl methylcellulose, hyetellose, hypromellose, hyaluronic acid, a derivative, wherein the derivative is an acid, salt or ester of these compounds, or a combination of the aforementioned substances.

11. The method according to claim 1, characterized in that the preparation is an aqueous solution.

12. The method according to claim 1, characterized in that the preparation is provided in the form of a solution, irrigation, suspension, ointment, cream, lotion, paste, spray, jelly, aerosol, nasal spray or nose drops.

13. The method according to claim 1, characterized in that it is provided in the form of an isotonic or hypertonic composition.

14. (canceled)

15. A method to prevent a secretion build-up, the occurrence of desiccation and/or inflammatory irritations of mucous membranes, comprising applying a preparation comprising at least one osmolyte to said membranes,
characterized in that the osmolyte is 1,4,5,6-tetrahydro-2-methyl-pyrimidine-4-carboxylic acid (ectoine), 4,5,6,7-tetrahydro-2-methyl-1H-[1,3]-diazepine-4-S-carboxylic acid (homoectoine), S,S-β-hydroxy-1,4,5,6-tetrahydro-2-methyl-pyrimidine-4-carboxylic acid (hydroxyectoine), di-myo-inositol phosphate (DIP), cyclic 2,3-diphosphoglycerate (cDPG), 1,1-di-glycerol phosphate (DGP), β-mannosylglycerate (firoin), β-mannosylglyceramide (firoin-A), di-mannosyl-di-inositol phosphate (DMIP), glucosylglycerol and/or an acid, salt or ester, of said compounds.

16. A method Use according to claim 14, characterized in that the agent serves to reduce the formation of edemas and/or improve the nasal ventilation comprising applying a preparation comprising at least one osmolyte to said membranes.
characterized in that the osmolyte is 1,4,5,6-tetrahydro-2-methyl-pyrimidine-4-carboxylic acid (ectoine), 4,5,6,7-tetrahydro-2-methyl-1H-[1,3]-diazepine-4-S-carboxylic acid (homoectoine), S,S-β-hydroxy-1,4,5,6-tetrahydro-2-methyl-pyrimidine-4-carboxylic acid (hydroxyectoine), di-myo-inositol phosphate (DIP), cyclic 2,3-diphosphoglycerate (cDPG), 1,1-di-glycerol phosphate (DGP), β-mannosylglycerate (firoin), β-mannosylglyceramide (firoin-A), di-mannosyl-di-inositol phosphate (DMIP), glucosylglycerol and/or an acid, salt or ester, of said compounds.
